(19) United States
(12) Patent Application Publication
(10) Pub. No.: US 2026/0331127 A1
(43) Pub. Date: Oct. 22, 2026

(54) SYSTEM AND METHOD FOR EMOTIONAL INTELLIGENCE MAPPING VIA VOICE-ACTIVATED CHAKRA RESONANCE AND BIOMETRIC FEEDBACK

(71) Applicant: Satyan MISHRA, Maharashtra (IN)

(72) Inventor: Satyan MISHRA, Maharashtra (IN)

(73) Assignee: Satyan MISHRA, Maharashtra (IN)

(21) Appl. No.: 19/651,844

(22) Filed: Apr. 20, 2026

(30) Foreign Application Priority Data
Apr. 21, 2025 (IN) 202521037914

(51) Int. Cl. G10L 25/63; G10L 15/02; G10L 15/04; G10L 15/183

(52) U.S. Cl. CPC G10L 25/63; G10L 15/02; G10L 15/04; G10L 15/183

(57) ABSTRACT

The present disclosure discloses on a system and method for assessing personality, well-being and emotional intelligence (EQ) parameters of an individual. The method comprises receiving voice input from a user via an audio input module, capturing, via a context acquisition module, contextual metadata associated with the voice input, the metadata including at least a time of day, a gender and age of the user, and a geographic region, segmenting the voice input into discrete acoustic features, mapping said acoustic features onto a plurality of personality traits and identifying the corresponding chakra energy levels the traits correspond to, via chakra resonance mapping. The invention discloses aggregating the scores from mapping, preparing a multidimensional Emotional Quotient (EQ) profile based on the extracted acoustic features, the contextual metadata, and optionally the biometric feedback/parameters, and generating a structured text-based report summarizing EQ parameters and personality traits associated with the individual.

### CROSS REFERENCING RELATED APPLICATIONS AND PRIORITY DETAILS

This application claims the benefit of and priority to Indian Provisional Application No. 202521037914 filed on Apr. 21, 2025.

### TECHNICAL FIELD

The present disclosure relates to a system and method that uses voice input, contextual data and optionally, biometric feedback to evaluate, quantify, and improve emotional intelligence. The present disclosure integrates acoustic processing from a voice input sample in a plurality of languages with chakra-based energetic mapping to derive an emotional intelligence and wellness score and guide personalized healing interventions.

### BACKGROUND OF THE INVENTION

The following description of related art is intended to provide background information pertaining to the field of invention. This section may include certain aspects of the art that may be related to various aspects of the present disclosure. However, it should be appreciated that this section be used only to enhance the understanding of the reader with respect to the present disclosure, and not as admissions of prior art.

Emotional intelligence (EQ) and personality assessment have traditionally relied on questionnaires, self-reporting, or observational methods. These approaches often suffer from subjectivity, generalizations, cultural bias, and lack of reproducibility, limiting their effectiveness in providing actionable insights.

Existing emotional intelligence and emotion-recognition systems rely heavily on semantic content, predefined emotion labels, or visual cues. Such systems fail to capture emotional capacity, regulation, and structural emotional coherence, particularly in audio-only or low-resource contexts. While, advances in speech processing and voice biomarker research have introduced methods for detecting stress, emotion, or health conditions from acoustic features. However, most existing systems focus on single-label outputs, such as “stress” or “happiness,” without offering a structured, multidimensional profile of personality or emotional intelligence.

Prior art in voice biomarker analysis, such as U.S. Pat. No. 7,222,075B2 and US20210219893A1, demonstrates the potential of acoustic features like pitch, harmonics, and cepstral coefficients for health screening. Yet these systems lack integration with holistic frameworks that connect voice resonance to broader well-being attributes.

Meditation and wellness applications have attempted to incorporate chakra-based models, often using questionnaires or guided content. These solutions, however, do not ground their outputs in quantitative acoustic evidence, resulting in generalized recommendations rather than personalized, evidence-based guidance.

Personality trait prediction systems, such as US20180137432A1, have explored correlations between voice features and psychological attributes. Nonetheless, these systems typically depend on labeled datasets and machine learning models that may not generalize across languages, accents, or recording conditions.

A key limitation of existing technologies is the absence of traceable evidence and confidence measures. Users often receive unreliable outputs and generalized conclusions derived subjectively, reducing trust, accuracy and limiting adoption in sensitive contexts like wellness or coaching and also lacking information on energy misalignments or problem areas.

Furthermore, most current systems do not integrate biometric signals such as breath metrics. This omission prevents validation of energetic states and reduces the reliability of voice-only interpretations.

There remains a gap between quantitative physiological monitoring and holistic well-being interventions in existing solutions. No existing model integrates voice-based analysis, and vibrational mapping of acoustic features from user's voice inputs, relying on contextual metadata and optionally on biometric feedback, to provide structured emotional intelligence profiling. Thus, there exist a need in the art to provide a technique which overcomes the above-mentioned problems, system and method for emotional intelligence mapping via voice-activated chakra resonance and biometric feedback.

### SUMMARY OF THE INVENTION

This section is provided to introduce certain objects and aspects of the disclosed methods and systems in a simplified form that are further described below in the detailed description. This summary is not intended to identify the key features or the scope of the claimed subject matter.

To alleviate the shortcomings of existing methods and systems, as discussed in the background section, the present disclosure addresses these limitations by introducing a computer-implemented system that extracts a plurality of acoustic features from a voice input, maps them on to the energy-parameters associated with various chakra levels via a chakra-based resonance model, and synthesizes a multidimensional personality, EQ and wellness profile, specific to each user. This profile is enriched with a plurality of contextual metadata and may optionally be validated by biometric signals.

Initially, the mapping of acoustic features is carried out in view of the contextual metadata to identify the key traits of the user, and thereafter, the identified key traits are mapped on to the chakra-based energy parameters/attributes based on the resonance, to obtain a personality and EQ score. The mapping and scoring is done for presence, stability, overactivation, or suppression among other traits of the user. These scores are aggregated at the chakra level to derive emotional capacity, balance, and regulation metrics, among others.

By combining structured acoustic evidence, contextual metadata and optionally, biometric validation from modern science, with chakra-based energy resonance mapping from knowledge of ancient science and culture, an automated machine-readable structured report is generated providing a repeatable, transparent, and personalized method for assessing personality and emotional intelligence. This integration bridging modern digital signal processing with ancient energetic frameworks offers a novel pathway for holistic persona and emotional well-being assessment not known in the art.

In one non-limiting embodiment of the present disclosure, a method for assessing personality and emotional intelligence (EQ) parameters of an individual is disclosed. The method comprises receiving voice recording data (a voice input) from a user via an audio input module, and capturing, via a context acquisition module, the contextual metadata associated with the voice input and/or the user, the said metadata including one or more from a time of day, a gender of the user, an age of the user, and a geographic region. The method further comprises segmenting (or splitting) via an acoustic analysis module, the voice input into a plurality of discrete acoustic frequency blocks and therefrom extracting a plurality of acoustic features including but not limited to spectral harmonics, pitch stability, breath silence ratio, spectral bandwidth, spectral ratio, spectral mean, spectral contrast, spectral flatness, segment durations, timbre, MFCCs, spectral flatness, harmonic ratios, and breath/silence metrics etc.

According to an embodiment, the said method further comprises normalizing the voice input received as is and then, in view of the contextual metadata. The method further comprises mapping the plurality of acoustic features, once normalized, wherein said mapping comprises identification of a plurality of personality traits based on the acoustic features segmented, followed by determining a corresponding chakra level based on the personality traits mapped, using chakra-based resonance mapping The method thereafter comprises aggregating the results/scores from the mapping and preparing a multidimensional Emotional Quotient (EQ) profile inferred from the mapping steps and accordingly, generating a structured text-based report summarizing EQ parameters and personality traits associated with individual/ user, based on the multidimensional EQ profile.

In another non-limiting embodiment of the present disclosure, the method further comprises optionally further capturing biometric parameters of the user from a wearable device or any other device with one or more sensors, wherein, the biometric parameters may include one or more from heart rate variability (HRV), breath metrics and other such biometric inputs, to validate energetic states associated with the extracted acoustic features, and preparing the multidimensional EQ profile further based on the biometric parameters.

In another non-limiting embodiment of the present disclosure, the mapping further comprises determining the specific t chakra levels across the seven primary energy centers indexes which correspond to the personality traits identified during the acoustic feature mapping, said chakra levels including EQ-R to EQ-U, and wherein the EQ-R to EQ-U indexes comprise EQ-R (Root), EQ-F (Flow), EQ-C (Confidence), EQ-E (Empathy), EQ-X (Expression), EQ-I (Insight), and EQ-U (Unity).

In another non-limiting embodiment of the present disclosure, the multidimensional EQ profile is based the mapping and aggregating the scores/results by an inference and aggregation module that preferably utilizes a Large Language Model (LLM) constrained by a prompt template that enforces interpretation grounded strictly in the numeric acoustic features and the captured contextual metadata, and optionally, the biometric data/feedback.

In another non-limiting embodiment of the present disclosure, the method further comprises suggesting personalized wellness recommendations comprising specific—mantras, chants or practices, based on the detection of dormant or active energy states within the synthesized EQ profile.

In yet another non-limiting embodiment of the present disclosure, a voice-based assessment system for inferring personality, well-being and emotional intelligence (EQ) parameters is disclosed. The system comprises an audio input module configured to receive voice recording data/voice input from a user and normalize the voice input for background noise, interception anomalies, disturbances, device quality etc.. The system further comprises a context acquisition module configured to capture and contextual metadata associated with the voice input, the contextual metadata including one or more from a time of day, a gender, an age and a geographic region. The system also comprises an acoustic analysis module configured to segment the voice input into a plurality of discrete acoustic frequency blocks and therefrom extract a plurality of acoustic features including spectral harmonics, pitch stability and the like. The system further comprises an inference and aggregation module configured to map a plurality of personality traits with the acoustic features, normalized from the voice input and in view of the contextual metadata. The inference and aggregation module is further configured to determine the corresponding chakra levels/parameters based on the personality traits so mapped using a chakra-based resonance mapping. Additionally, the said module aggregates the results/scores from the mapping and prepares a multidimensional Emotional Quotient (EQ) profile. The system also comprises an output module configured to generate a structured text-based report summarizing EQ parameters and personality traits associated with individual, based on the multidimensional EQ profile.

In yet another non-limiting embodiment of the present disclosure, the acoustic features comprise a dictionary of numeric descriptors-direct or derived-including the spectral harmonics, pitch stability Mel-frequency cepstral coefficients (MFCCs), spectral flatness, breath silence ratio, spectral bandwidth, spectral ratio, spectral mean, spectral contrast, spectral flatness, segment durations, timbre, and harmonic ratios.

In yet another non-limiting embodiment of the present disclosure, the output module is configured to convert the determined multidimensional EQ profile into a human-readable artifact in a format selected from Markdown, HTML, or PDF format, or any other format known to a person skilled in the art.

In yet another non-limiting embodiment of the present disclosure, the system further comprises an interactive follow-up module utilizing an agentic workflow to maintain chat history and answer user queries grounded specifically in the generated report.

In yet another non-limiting embodiment of the present disclosure, the acoustic analysis module is further configured to compute a breath/silence ratio and fundamental frequency (F0) range to distinguish between different emotional intensities within the voice recording data/voice input.

The foregoing summary is illustrative only and is not intended to be in any way limiting. In addition to the illustrative embodiments, and features described above, further embodiments, and features will become apparent by reference to the drawings and the following detailed description.

### BRIEF DESCRIPTION OF DRAWINGS

The accompanying drawings, which are incorporated in and constitute a part of this disclosure, illustrate exemplary embodiments and, together with the description, serve to explain the disclosed principles. Also, the embodiments shown in the figures are not to be construed as limiting the invention, but the possible variants of the monitoring method and system/device according to the invention are illustrated herein to highlight the advantages of the invention. Some embodiments of system and/or methods in accordance with embodiments of the present subject matter are now described, by way of example only, and with reference to the accompanying Figures.

FIG. 1 illustrates an exemplary environment for a voice-based emotional quotient (EQ) assessment, in accordance with the embodiments of the present disclosure;

FIG. 2 illustrates a flow chart of a method for assessing personality and emotional intelligence (EQ) parameters of an individual, in accordance with an embodiment of the present disclosure.

FIG. 3 presents a voice-based assessment system for inferring personality and emotional intelligence (EQ) parameters, in accordance with an embodiment of the present disclosure; and

It should be appreciated by those skilled in the art that any block diagrams herein represent conceptual views of the illustrative systems embodying the principles of the present subject matter. Similarly, it will be appreciated that any flowcharts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION OF THE EMBODIMENTS

The description in ensuing paragraphs cover exemplary embodiments of the present invention which are described in sufficient detail to enable those skilled in the art to practice the invention, but other embodiments may be utilized and logical, mechanical, electrical, and other changes may be made without departing from the scope of the present invention. It will be apparent, however, that the disclosed embodiments may be practiced without these specific details. Therefore, following detailed description is not to be taken in a limiting sense, and the scope of the present invention is defined only by the appended claims.

Several features described hereafter can each be used independently of one another or with any combination of other features. However, any individual feature may not address any of the problems discussed above or might address only some of the problems discussed above. Some of the problems discussed above might not be fully addressed by any of the features described herein.

While the disclosure is susceptible to various modifications and alternative forms, specific embodiments thereof have been shown by way of example in the drawings and will be described in detail below. It should be understood, however, that these embodiments are not intended to limit the disclosure to the particular form disclosed, but on the contrary, the disclosure is to cover all modifications, equivalents, and alternatives falling within the spirit and the scope of the disclosure.

Although definitions of all terms coined and/or used in describing the present invention have been elaborately described in the specification; however, all terms not specifically described shall be given the meaning most obvious and apparent to a person having ordinary skilled in the art.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise(s)”, “comprising”, “include(s)”, or any other variations thereof, are intended to cover a non-exclusive inclusion, such that a setup, device, apparatus, system, or method that comprises a list of components or steps does not include only those components or steps but may include other components or steps not expressly listed or inherent to such setup or device or apparatus or system or method. In other words, one or more elements in a device or system or apparatus proceeded by “comprises . . . a” does not, without more constraints, preclude the existence of other elements or additional elements in the system.

The expressions like “at least one” and “one or more” may be used interchangeably or in combination throughout the description.

Accordingly, the term “module” or “model” should be understood to encompass a tangible entity, be that an entity that is physically constructed permanently configured (hardwired) or temporarily configured (programmed) to operate in a certain manner and/or to perform certain operations described herein.

Accordingly, the terms “Artificial intelligence (AI) model” or “AI Model”, “Machine Learning Model” and “ML Model” or “system” should be understood to encompass a tangible entity, be that an entity that is physically constructed permanently configured (hardwired) or temporarily configured (programmed) to operate in a certain manner and/or to perform certain operations described herein.

It should be understood that the modules illustrated and described herein, including but not limited to the context acquisition module, inference and aggregation module, acoustic analysis module, audio input module, output module, and processing unit, are presented for purposes of explanation and example only. The designation of a component as a “module” does not imply any particular hardware or software implementation. Each module may be embodied in dedicated circuitry, programmable logic, firmware, software executed by a general-purpose processor, or any combination thereof.

In one non-limiting aspect, the one or more modules may comprise a dedicated hardware component known to a person skilled in the art, which are required for performing the method and functionalities discussed in below embodiments.

Further, the functional separation of modules in the figures is not intended to require physical separation. A single hardware or software element m ay perform the functions of multiple modules, or conversely, a single module's functionality may be distributed across multiple elements. The specific arrangement shown is merely illustrative and should not be construed as limiting.

Accordingly, references to modules are intended to encompass any suitable structural or functional implementation capable of performing the recited operations, and variations, substitutions, or integrations of such modules fall within the scope of the present disclosure.

The present disclosure addresses these limitations by introducing a computer-implemented pipeline/system that extracts a set of acoustic features, maps them to chakra levels via a chakra energy-based resonance model, and synthesizes a multidimensional EQ profile. The invention combines language-independent voice resonance analysis, structural emotional architecture, chakra-level mapping, and capacity-based emotional intelligence scoring while accounting for various contextual variations in input types, user particulars and locations.

The invention discloses a voice-based assessment system for inferring personality, well-being and emotional intelligence (EQ) parameters of a user, said the system comprising an audio input module configured to receive a voice input from the user and normalize the said voice input. The system further comprises a context acquisition module configured to capture a plurality of contextual metadata associated with the voice input and the user, wherein the contextual metadata comprising at least one of a time of day, a gender, an age, and a regional location.

The system further comprises an acoustic analysis module configured to segment the voice recording data into discrete acoustic frequency blocks and extract a plurality of acoustic features. According to an embodiment of the invention the acoustic analysis module is further configured to compute a breath/silence ratio and a fundamental frequency (F0) range to distinguish between different emotional intensities within the voice recording data.

According to the invention, the system includes an inference and aggregation module configured for map the plurality of acoustic features, such that said module first identifies a plurality of personality traits based on the acoustic features segmented, and then, determines a corresponding chakra level based on the personality traits mapped, using chakra-based resonance mapping, wherein further, said modules is conjured to aggregate the scores from the mapping and prepare a multidimensional Emotional Quotient (EQ) profile.

Further, the system additionally comprises an output module configured to generate a structured text-based report summarizing EQ parameters and personality traits associated with the user, based on the multidimensional EQ profile. According to a non-limiting embodiment of the invention, the output module is configured to convert the determined multidimensional EQ profile into a human-readable artifact in a format selected from Markdown, HTML, or PDF.

In yet another embodiment of the invention, the system further comprises an interactive follow-up module utilizing an agentic workflow to maintain chat history and answer user queries grounded specifically in the generated report.

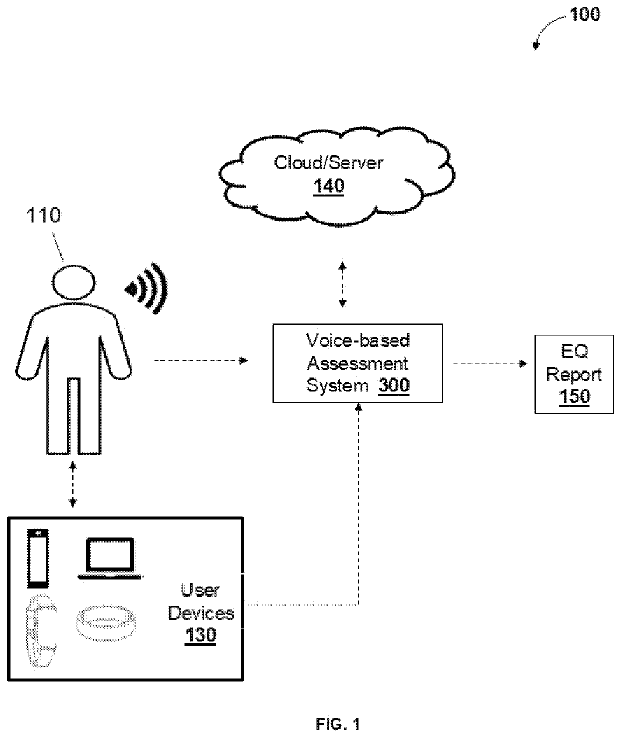

FIG. 1 illustrates an exemplary environment for a voice-based emotional quotient (EQ) assessment, in accordance with one of the embodiments of the present disclosure.

The environment 100 comprises a user 110 with at least one user device 130, voice-based assessment system 300, a cloud/server 140 in communication with each other for emotional quotient (EQ) assessment. The user 110 may require to assess emotional quotient (EQ) traits comprising the user's persona-related attributes and/or wellbeing-oriented “energy” indicators from a voice recording (voice input) including but not limited to speech/chant/mantra or a given excerpt.

The user device 110 may include, for example, a mobile phone, a laptop, a tablet, a desktop computer, and/or the like. Further, the user device 110 may comprise smart band, smart ring, smart watch for monitoring real-time health related features. However, the user device 110 is not limited to above examples, and may include any other device known to a person skilled in the art for inputting voice recording data for well-being and EQ assessment. In an embodiment, the user device 130 may be used to capture one or more of contextual metadata and/or biometric data for more specific, personalized and accurate assessment.

In one non-limiting embodiment of the present disclosure, the user device 130 may be communication with smart wearable device worn by the user 110. The smart wearable device may include, but not limited to, smart watches, smart rings, fitness trackers, ear phones, smart glasses, and smart clothing, etc. The voice-based assessment system 300 may be in communication with the cloud/server 140 through a network may include, for example, the Internet, a private network, public network or other configuration that operates using any one or more known communication protocols such as, for example, transmission control protocol/Internet protocol (TCP/IP). Other examples of the communication protocols include Bluetooth®, BLE®, Wi-Fi, UWB, and cellular technologies such as Time Division Multiple Access (TDMA), Code Division Multiple Access (CDMA), High Speed Packet Access (HSPDA), Long-Term Evolution (LTE), Global System for Mobile Communications (GSM), and Fifth Generation (5G), to name a few.

The exemplary system environment 100 may assess personality and emotional intelligence (EQ) parameters of an individual/user using voice-based input and contextual metadata. The user 110 who provides voice recording data through various user devices 130, including smartphones, laptops. Further smartwatches, and wearable rings may be used to capture biometric parameters such as heart rate variability (HRV) and breath metrics.

The voice recording data/voice input is received by a voice-based assessment system 300, which serves as the central processing unit for acoustic and contextual analysis. The voice-based assessment system 300 normalizes and preprocesses the received voice data and capture contextual metadata associated with the voice input, including time of day, gender, age and geographic region.

The voice-based assessment system 300 segments the voice recording into discrete acoustic features by first splitting the voice input into frequency blocks and then extracting the numeric acoustic features, direct and derived, such as spectral harmonics, pitch stability, breath silence ratio, spectral bandwidth, spectral ratio, spectral mean, spectral contrast, spectral flatness, segment durations, timbre, MFCCs, spectral flatness, harmonic ratios, and breath/silence metrics.etc.. These features are computed using a digital signal processing stack by the acoustic analysis module.

The split and extracted acoustic features are mapped to the personality trait patterns via phoneme resonance, for instance, and the identified personality traits are then used to determine which chakra-wise energy centers do they correspond to through chakra-based resonance mapping, as know from the yogic/ancient scriptures. The mapping process utilizes resonance model corresponding to numerous chakra levels across seven primary indexes: EQ-R to EQ-U. The indexes represent Root (EQ-R), Flow (EQ-F), Confidence (EQ-C), Empathy (EQ-E), Expression (EQ-X), Insight (EQ-I), and Unity (EQ-U).

The voice-based assessment system 300 applies a constrained prompt template to a Large Language Model (LLM), ensuring interpretations are grounded strictly in acoustic features and contextual metadata, and optionally, the biometric parameters/feedback. The LLM generates a multidimensional EQ profile comprising chakra-wise scores, an overall EQ score, and personality trait indicators.

The output is formatted into a structured text-based report, which may be rendered in Markdown, HTML, or PDF formats. The report includes interpretive tables, energy center mappings, and personalized wellness recommendations based on detected energetic states. Recommendations may include specific mantras/chants or practices/rituals tailored to dormant or active chakra states, enhancing the user's emotional balance.

The voice-based assessment system 300 optionally integrates biometric data from wearable devices, such as heart rate variability (HRV) and breath metrics, to validate acoustic inferences. In an embodiment of the invention, these biometric parameters are fused with voice features to refine the EQ profile and improve interpretive accuracy.

The voice-based assessment system 300 communicates with a cloud/server 140 for data storage, model access, and report synchronization. The final output, labeled as EQ Report 150, is delivered to the user via their connected user devices 130, enabling review and follow-up.

The voice-based assessment system 300 maintains chat history and allows the user to ask questions grounded in the generated report. Each follow-up response is conditioned on the original analysis, ensuring consistency and avoiding speculative drift. The system 300 supports multiple LLM providers, allowing flexibility in deployment while maintaining a fixed feature extraction pipeline.

FIG. 1 thus encapsulates the end-to-end flow: from voice input and contextual capture to feature extraction, inference, report generation, and interactive feedback. The architecture supports both offline and cloud-based operation, enabling secure and scalable deployment across diverse user environments.

Overall, FIG. 1 represents a novel integration of acoustic analysis, chakra mapping, structured reporting, and agentic interaction for voice-based EQ assessment.

The technical effect of the disclosure is to enable a structured and context-aware assessment of an individual's emotional intelligence (EQ) and personality traits using only voice input and optionally biometric data. By extracting a set of numeric acoustic features—such as spectral harmonics, pitch stability, breath silence ratio, spectral bandwidth, spectral ratio, spectral mean, spectral contrast, spectral flatness, segment durations, timbre, MFCCs, spectral flatness, harmonic ratios, and breath metrics—and grounding the interpretive output in these features via a constrained prompt to a Large Language Model (LLM), the system ensures that the analysis remains evidence-based and reproducible. This approach eliminates reliance on subjective questionnaires or free-form speculation, enhances accuracy, personalization, and allows for automated generation of chakra-wise EQ profiles and personalized wellness recommendations, thereby improving the reliability, scalability, and usability of voice-based emotional assessment systems.

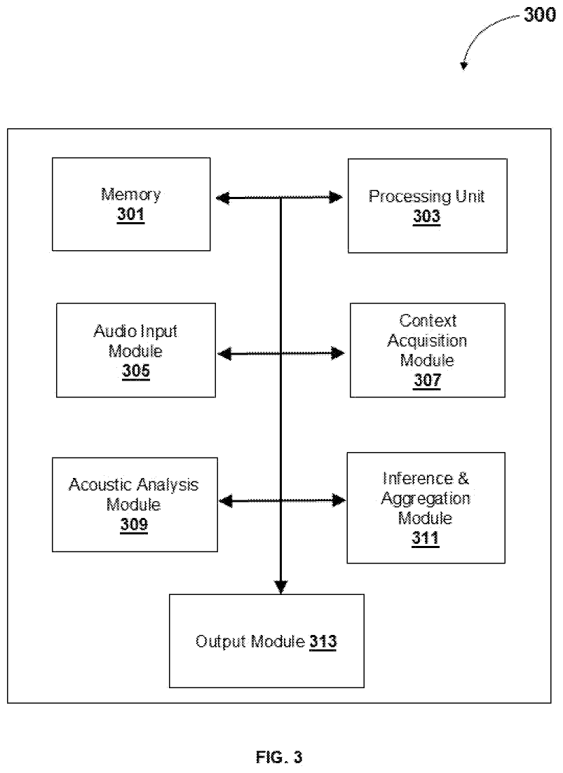

FIG. 3 illustrates the system architecture of the voice-based assessment system 300 for inferring personality and emotional intelligence (EQ) parameters, in accordance with an embodiment of the present disclosure.

In an aspect of the present disclosure, the voice-based assessment system 300 may comprise a memory 301, a processing unit 303, an audio input module 305, context acquisition module 307, an acoustic analysis module 309, an inference & aggregation module 311, and an output module 313 communicatively coupled with each other. The voice-based assessment system 300 may be in communication with the cloud/server 140 for retrieving data stored at the cloud/server 140. In one non-limiting embodiment, the voice-based assessment system 300 may comprise a wearable or computing device 130 comprising the memory 301 and the processing unit 303.

The voice-based assessment system 300 is a computer-implemented architecture designed to process voice recordings input, acquire contextual metadata, compute acoustic features, and generate structured EQ and personality reports. The system 300 integrates multiple modules, each responsible for a distinct stage of the workflow, ensuring grounded, repeatable, and structured inference of wellbeing attributes.

The memory 301 stores both temporary and persistent data required for the assessment process. This includes the computed acoustic feature dictionary (e.g., RMS energy, MFCCs, spectral descriptors, pitch stability, harmonic ratio), contextual metadata (time of day, gender, geographic region), and generated artifacts (Markdown, HTML, PDF reports). Memory also maintains historical user data for trend analysis and supports the agentic follow-up Q&A loop by retaining chat history and contextual grounding. In alternative embodiments, the memory 301 may be implemented as volatile RAM, persistent disk storage, or cloud-based repositories. The choice of memory architecture does not limit the scope of the present disclosure.

The processing unit 303 executes the computational steps of the system 300. It orchestrates audio pre-processing, feature extraction, inference, and report generation. The processing unit 303 integrates digital signal processing libraries (e.g., librosa, numpy) with large language model (LLM) providers (e.g., OpenAI-compatible or Google Gemini SDK). It ensures that interpretations are strictly grounded in the acoustic features and contextual metadata, enforcing a constrained-output schema for consistency and traceability. In one non-limiting embodiment, various functionalities of different modules, as discussed in below embodiments, may be performed by the processing unit 303. The processing unit 303 may be implemented using microcontrollers, distributed computing clusters, or specialized AI accelerators. Any hardware capable of executing the described workflow falls within the scope of the present disclosure.

The audio input module 305 receives the user's voice recording input, which may be speech, chant, or mantra, preferably in an audio file path (ex. WAV/MP3). The audio is normalized to a fixed sampling rate (e.g., 22,050 Hz) to ensure uniformity across devices and environments. This module 305 validates the input, terminating the process if the audio is empty or unreadable. It prepares the recording for segmentation into discrete acoustic features. The audio input may originate from telephony systems, wearable devices, or cloud-based voice APIs and the sources may include mobile phone recording, web upload, call recording, etc. The present disclosure is not limited to any particular input source.

The context acquisition module 307 captures contextual metadata associated with the voice recording/voice input. This may include, but not limited to, time of day, gender, age and geographic region. The system 300 further supports this by noting that contextual grounding improves interpretive accuracy. This metadata is later combined with acoustic features to generate a multidimensional EQ profile, ensuring that persona inference is sensitive to situational and demographic factors. Contextual metadata may be derived from user-entered information, sensor data, or external databases. The present disclosure encompasses all such variations.

The acoustic analysis module 309 segments the voice recording/voice input into a plurality of discrete acoustic features. The segmenting by acoustic analysis module 309 comprises splitting the voice input into a plurality of discrete acoustic frequency blocks, and extracting the plurality of acoustic features from said frequency blocks split. These acoustic features include spectral harmonics, pitch stability, MFCCs, spectral centroid, bandwidth, roll-off, harmonic ratio, breath/silence ratio, and fundamental frequency (F0) range etc..

In an exemplary embodiment, the audio signal from the voice input is processed to split and extract meaningful characteristics from sound recordings. They're typically used for audio analysis, music classification, speech processing, and machine learning tasks such as
a. Loudness & Energy Features including but not limited to
   i. RMS Mean (Root Mean Square) which is the average loudness level of the audio across the entire file, measured in decibels (dB). It tells us how “loud” the audio is overall.
   ii. RMS Std which is variation in loudness over time. A high value means the volume fluctuates a lot; a low value means it's consistent.
b. Speech/Breath Features including but not limited to Breath Silence Ratio with refers to proportion of silence or breath sounds relative to the total audio. Useful for detecting speech patterns or identifying breathing pauses in voice recordings.
c. Timbral/Frequency Features including but not limited to
   i. MFCC Mean & Std—Mel-Frequency Cepstral Coefficients. These capture the “timbre” or character of the sound—how it sounds beyond just pitch or loudness. MFCC means are the average values; MFCC stds show variation.
   ii. Spectral Centroid Mean & Std refers to where the “center of mass” of the frequency spectrum is located. A higher value means the sound is brighter/more treble-heavy; lower means it's darker/bassier. The std measures how this shifts.
   iii. Spectral Bandwidth Mean & Std refer to how spread out the frequencies are. Narrow bandwidth=pure tones; wide bandwidth=noisy or complex sounds.
   iv. Spectral Rolloff Mean refers to the frequency where 85% of the audio energy is below it. Helps distinguish bright sounds from dull ones.
   v. Spectral Contrast Mean refers to the difference in power between peaks and valleys in the frequency spectrum. Higher values indicate more dynamic, varied sounds.
   vi. Spectral Flatness Mean refers to how “flat” the frequency distribution is (0=purely tonal, 1=like white noise). Measures whether the sound is smooth or harsh.
d. Temporal Features including but not limited to
   i. Zero Crossing Rate (ZCR) Mean referring to how often the audio signal crosses zero (changes from positive to negative). High ZCR typically indicates noisy or consonant-heavy audio; low ZCR suggests pitched tones. Useful for speech/music distinction.
   ii. Segment Durations refers to lengths of individual audio segments or frames being analyzed. Tells you how the audio was broken up into chunks for analysis.

These acoustic features allow compression of complex audio information into simple numbers that machine learning models can process. Instead of analyzing raw waveforms (which are huge), the system can extract these plurality of acoustic features that capture the essential characteristics of the sound, making it much faster to train models for tasks like classification, anomaly detection, or speaker identification

The present disclosure describes that these feature descriptors form a “features dictionary” suitable for downstream inference, mapping them to specific personality traits based on, for instance, phoneme-resonance patterns, which in turn is mapped onto the chakra-based energy parameters by chakra resonance mapping for EQ profiling. Acoustic features may be computed using alternative algorithms, including proprietary or machine-learning-based feature extraction methods. The present disclosure is not restricted to the specific descriptors listed.

The inference & aggregation module 311 determines a multidimensional EQ profile using the segmented (split and extracted acoustic features) and captured contextual metadata. This module 311 is configured to map the plurality of acoustic features, such that said module 311 first identifies a plurality of personality traits based on the acoustic features segmented, and then, determines a corresponding chakra level based on the personality traits mapped, using chakra-based resonance mapping. The module 311 employs an inference engine constrained by a prompt template to ensure that interpretations remain grounded in numeric evidence and contextual data. The module 311 further aggregates the scores from the mapping done and prepare a multidimensional Emotional Quotient (EQ) profile.

According to an embodiment, the prompt template is constructed such that it defines the system role (voice-based chakra/persona analysis), provides “feature hints” mapping each numeric acoustic feature to interpretive axes (energy, stability, harmonicity, brightness, breath), enforces an output structure (e.g., a fixed Markdown report template and/or a structured result object), and/or appends the computed feature dictionary and contextual metadata, optionally biometric parameters, to ensure all conclusions are grounded.

The system 300 specifies that the output includes chakra-wise scores (0-10), an overall EQ score, confidence levels, and interpretive notes. Biometric parameters such as HRV and breath metrics may also be integrated to validate energetic states in a non-limiting embodiment. The inference engine may employ deterministic rule-based algorithms, statistical models, or hybrid AI approaches. The present disclosure covers all inference techniques that remain grounded in numeric evidence.

The output module 313 generates a structured text-based reports summarizing EQ parameters and personality traits. As claimed, the output may be converted into human-readable artifacts in Markdown, HTML, or PDF formats. The system 300 may, in an embodiment, support automatic artifact generation and storage, as well as an interactive follow-up module that maintains chat history and answers user queries grounded in the generated report. This ensures continuity, personalization, and iterative refinement of the user's wellbeing profile. Output formats may include dashboards, mobile notifications, or API endpoints. Interactive follow-up may be implemented via chatbots, agentic workflows, or voice assistants. The present disclosure is not limited to any particular output modality.

The embodiments described herein are illustrative examples provided for clarity. Variations in hardware, software, algorithms, and data sources may be employed without departing from the scope of the claims. References to specific libraries, formats, or biometric parameters are exemplary and not limiting. The present disclosure encompasses all equivalent implementations that achieve the described functionality of voice-based persona and EQ assessment.

It may be noted that, in some embodiments, the system 300 may include more or fewer components than those depicted herein. The various components of the system 300 may be implemented using hardware, software, firmware or any combinations thereof. Further, the various components of the system 300 may be operably coupled with each other. More specifically, various components of the system 300 may be capable of communicating with each other using communication channel media.

In one embodiment, the processing unit 303 may be embodied as a multi-core processor, a single core processor, or a combination of one or more multi-core processors and one or more single core processors. For example, the processing unit 303 may be embodied as one or more of various processing devices, such as a coprocessor, a microprocessor, a controller, a digital signal processor (DSP), a processing circuitry with or without an accompanying DSP, or various other processing devices including, a microcontroller unit (MCU), a hardware accelerator, a special-purpose computer chip, or the like.

In one embodiment, the memory 301 is capable of storing machine executable instructions, referred to herein as instructions. The instructions may be written in Python language or any other language know to a person skilled in the art. In an embodiment, the processing unit 303 is embodied as an executor of software instructions. As such, the processing unit 303 is capable of executing the instructions stored in the memory 301 to perform one or more operations described herein.

The memory 301 can be any type of storage accessible to the processing unit 303 to perform respective functionalities. For example, the memory 301 may include one or more volatile or non-volatile memories, or a combination thereof. For example, the memory 301 may be embodied as semiconductor memories, such as flash memory, mask ROM, PROM (programmable ROM), EPROM (erasable PROM), RAM (random access memory), etc. and the like.

According to an embodiment, the system comprises a trend analysis module that evaluates longitudinal changes in EQ indices and chakra activations. By comparing current results with historical data stored in the user database, the system identifies patterns, improvements, or deteriorations in wellbeing attributes. The present disclosure highlights that this capability supports iterative refinement and enables users to track progress over time, making the system suitable for ongoing wellbeing monitoring.

According to yet another embodiment, the results are integrated into the Dashboard & Cloud Sync. The structured EQ report, recommendations, and trend data are displayed on a user-facing dashboard and optionally synchronized with cloud storage for secure access across devices. In an embodiment, generated artifacts (Markdown, HTML, PDF) can be stored locally and synced to the cloud, ensuring both traceability and accessibility. This step also supports interactive follow-up Q&A sessions grounded in the generated report.

The present disclosure describes is a computer-implemented system and method for deducing a person's persona-related attributes and/or wellbeing-oriented “energy” indicators from a voice recording (speech/chant/mantra). The method takes an input audio recording, computes a set of acoustic features, and generates a structured analysis (including center/chakra-wise interpretation, an overall score, and suggested practices). The output is provided as a machine-readable structure and/or a human-readable report (Markdown/HTML/PDF), with optional interactive follow-up Q&A grounded in the generated report.

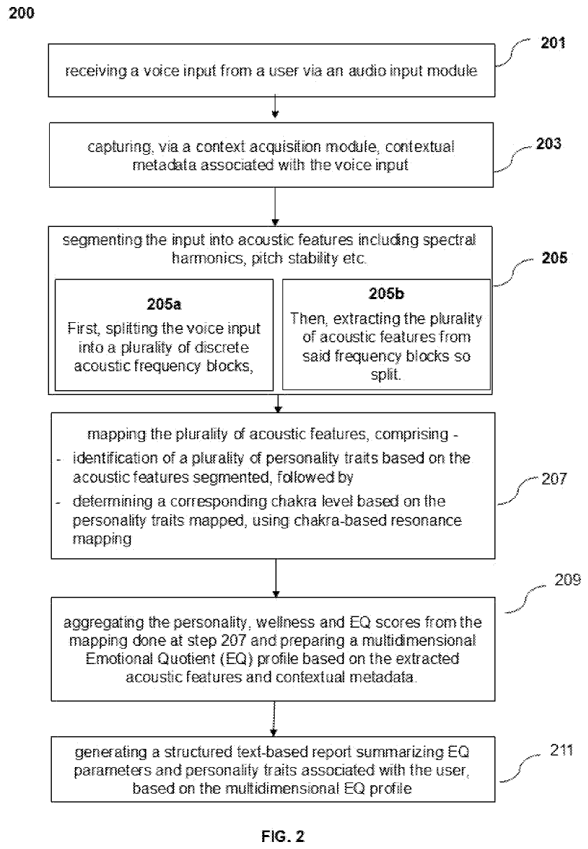

FIG. 2 illustrates a flow chart of a method 200 for assessing personality and emotional intelligence (EQ) parameters of an individual/a user, in accordance with an embodiment of the present disclosure.

At step 201, the system 300 receives voice recording data/input data from a user via an audio input module 305. The audio may be speech, chant, or mantra, captured through mobile devices, web uploads, or telephony systems. This input serves as the raw data for the system. The audio input is normalized to a fixed sampling rate (e.g., 22,050 Hz) to ensure consistency across diverse recording environments 100. This step establishes the foundation for subsequent acoustic feature splitting and extraction.

Standardizing (normalizing) the input ensures uniformity of acoustic descriptors, reduces variability due to device or environment, and enables reliable downstream feature extraction.

At step 203, the system 300 captures contextual metadata via a context acquisition module 307, including time of day, gender, age, and geographic region. As claimed, this metadata provides situational and demographic grounding for the analysis. The contextual grounding improves interpretive accuracy by aligning acoustic features with external conditions.

Incorporating metadata enhances personalization, reduces bias, and allows the EQ profile to reflect both vocal evidence and contextual influences.

At step 205, the system 300 segments the voice recording/voice input/audio into discrete acoustic features. More specifically, at step 205a the acoustic analysis module 309 first splits the audio/voice input into discrete acoustic frequency blocks and then at step 205b the module 309 extracts from the frequency block a plurality of acoustic features, including spectral harmonics and pitch stability. The present disclosure includes direct or derive feature descriptors such as RMS energy, MFCCs, spectral centroid, bandwidth, rolloff, harmonic ratio, pitch stability breath/silence ratio, and fundamental frequency (F0) range computed by the acoustic analysis module 309. These features form a structured dictionary that represents the vocal characteristics of the speaker, ensuring that the analysis is grounded in measurable acoustic evidence rather than subjective interpretation.

In an embodiment, at step 205a the splitting is done by a phoneme analysis engine of the acoustic analysis module 309 processes the voice input by breaking it down into phonemes to extract acoustic descriptors. Feature extraction transforms raw audio into structured numeric evidence, enabling repeatable and traceable analysis.

At step 207, the system 300 translates the extracted acoustic features into chakra-wise or center-specific activations by mapping. The mapping step comprises first identifying a plurality of personality traits based on the acoustic features extracted in step 205b, and then, determines a corresponding chakra level based on the personality traits mapped, using chakra-based resonance mapping.

At step 209, the system 300 aggregates the wellness and EQ scores from the mapping done at step 207 and prepares a multidimensional Emotional Quotient (EQ) profile based on the extracted acoustic features and contextual metadata.

As disclosed in the claims, the inference and aggregation module 311 carries out Steps 207 and 209 employing a Large Language Model (LLM) constrained by a prompt template, ensuring interpretations remain grounded strictly in numeric evidence. Biometric parameters such as HRV and breath metrics may also be integrated to validate energetic states.

The system enforces a mapping between numeric features and interpretive axes such as energy, stability, harmonicity, brightness, and breath. This allows the system to generate a multi-center perspective rather than a single global label, thereby supporting finer-grained persona and wellbeing analysis.

The is computed by aggregating the chakra-wise activations into a structured emotional intelligence measure. The constrained-output schema enforces consistency, prevents speculative interpretations, and produces standardized chakra-wise scores (0-10), overall EQ scores, and confidence notes which is then combined into an overall EQ index. This ensures that the output is in a standardized format, repeatable, and suitable for downstream automation. Integration of biometric signals enhances robustness and physiological validity. Chakra mapping provides a multi-center perspective, allowing finer-grained wellbeing profiling beyond single-label emotion recognition.

At step 211, the system 300 generates a structured text-based report summarizing EQ parameters and personality traits based on the multidimensional EQ profile. The report may be produced in Markdown, HTML, or PDF formats, which may be stored on cloud, server, or disk for later retrieval. This ensures traceability and allows users to revisit their analysis results. Local storage also supports offline usability and secure handling of personal data.

The report further may include recommendations which include suggested practices, wellbeing guidance, or persona-related insights. The inference module 311 uses a structured prompt to ensure that all suggestions remain grounded in the computed features, avoiding generic or speculative advice. This step transforms raw acoustic analysis into actionable outcomes for the user. The system 300 also supports interactive follow-up Q&A grounded in the generated report, ensuring continuity and personalization.

In an embodiment, the system starts an interactive loop where the user can ask follow-up questions. Each follow-up response is generated using the original analysis report as context, the accumulated chat history, and the loop ends when the user stops asking questions or a max turn count is reached.

Structured reporting improves usability, enables downstream automation, and provides actionable insights. Artifact generation ensures traceability, while interactive follow-up enhances user engagement and iterative refinement.

In another non-limiting embodiment of the present disclosure, the steps of method 200 may be performed in an order different from the order described above.

In yet another embodiment, the method begins with a Voice+Biometric Input (201b), where the system receives both an audio recording (speech, chant, mantra) and biometric signals such as heart rate variability (HRV) or other physiological markers. The audio is normalized to a fixed sampling rate (e.g., 22,050 Hz) and processed to ensure consistency across devices. Biometric data is captured simultaneously, providing a dual evidence stream that enhances the robustness of persona and wellbeing inference. All subsequent steps involve voice based and biometric based evidence for analysis of holistic wellbeing.

In a preferred embodiment the system and method can be implemented in software (e.g., Python) on standard computing hardware. Example library stack includes digital signal processing/feature extraction: librosa, numpy; LLM provider integration: OpenAI-compatible chat completions and/or Google Gemini SDK; report conversion: markdown renderer+optional HTML/PDF converter; agentic workflow orchestration: a state graph runtime for step-wise execution, checkpointing, and interactive pausing, or any other alternative as known in the art.

It is to be understood that not necessarily all objectives or advantages may be achieved in accordance with any particular embodiment described herein. Thus, for example, those skilled in the art will appreciate that certain embodiments may be configured to operate in a manner that achieves or optimizes one advantage or group of advantages as taught herein without necessarily achieving other objects or advantages as may be taught or suggested herein.

In a non-limiting embodiment of the present disclosure, one or more non-transitory computer-readable media may be utilized for implementing the embodiments consistent with the present disclosure. A computer-readable media refers to any type of physical memory (such as the memory) on which information or data readable by a processor may be stored. Thus, a computer-readable media may store one or more instructions for execution by the at least one processor, including instructions for causing the at least one processor to perform steps or stages consistent with the embodiments described herein. The term “computer-readable media” should be understood to include tangible items and exclude carrier waves and transient signals. By way of example, and not limitation, such computer-readable media can comprise Random Access Memory (RAM), Read-Only Memory (ROM), volatile memory, nonvolatile memory, hard drives, Compact Disc (CD) ROMs, Digital Video Disc (DVDs), flash drives, disks, and any other known physical storage media.

Thus, certain aspects may comprise a computer program product for performing the operations presented herein. For example, such a computer program product may comprise a computer readable media having instructions stored (and/or encoded) thereon, the instructions being executable by one or more processors to perform the operations described herein. For certain aspects, the computer program product may include packaging material.

The terms “an embodiment”, “embodiment”, “embodiments”, “the embodiment”, “the embodiments”, “one or more embodiments”, “some embodiments”, and “one embodiment”, “other embodiment”, “yet another embodiment”, “non-limiting embodiment” mean “one or more (but not all) embodiments of the disclosure(s)” unless expressly specified otherwise.

The various exemplary logical blocks and modules described in connection with the embodiments disclosed herein can be implemented or executed by a machine such as a “processor”. The processor may be a microprocessor, but alternatively, the processor may be a controller, a microcontroller, or a state machine, or a combination thereof. The processor can include an electrical circuit configured to process computer executable instructions. In another embodiment, the processor includes an application specific integrated circuit (ASIC), a field programmable gate array (FPGA), or other programmable device that performs logical operations without processing computer executable instructions. The processor can also be implemented as a combination of computing devices, e.g., a combination of a digital signal processor (DSP) and a microprocessor, a plurality of microprocessors, one or more microprocessors in conjunction with a DSP core, or any other such configuration. Although described herein primarily with respect to digital technology, the processor may also include primarily analog components. For example, some or all of the signal processing algorithms described herein may be implemented by analog circuitry or mixed analog and digital circuitry. A computing environment may include any type of computer system, including, but not limited to, a computer system that is based on a microprocessor, mainframe computer, a digital signal processor, a portable computing device, a device controller, or a computing engine within the device.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

A description of an embodiment with several components in communication with each other does not imply that all such components are required. On the contrary, a variety of optional components are described to illustrate the wide variety of possible embodiments of the disclosed methods and systems.

Finally, the language used in the specification has been principally selected for readability and instructional purposes, and it may not have been selected to delineate or circumscribe the inventive subject matter.

ADVANTAGES OF THE DICLOSURE:
Grounded, feature-driven interpretation: The system computes a plurality of acoustic features and requires the inference and aggregation module to base conclusions strictly on these numeric features rather than free-form speculation.
Structured reporting: Output is generated in a fixed report structure (tables/sections) and/or structured result fields (scores, suggestions, confidence/notes where implemented), improving consistency and downstream usability.
Multi-center perspective: Instead of a single global label, the system supports center/chakra-wise scoring plus an overall score, enabling finer-grained guidance.
End-to-end automation: Includes automatic report generation (Markdown/HTML/PDF) and optional interactive follow-up Q&A grounded in the produced report.
Provides flexibility: The inference and aggregation module can be configured to use different LLM providers/models without changing the feature extraction pipeline.

I claim:

1. A method [200] for assessing personality, well-being and emotional intelligence (EQ) parameters of a user [110], the method [200] comprising:
receiving [201] a voice input from the user [110] via an audio input module [305];
capturing [203], via a context acquisition module [307], a contextual metadata associated with the voice input, said contextual metadata including at least from a time of day, a gender of the user [110], an age of the user and a geographic region;
segmenting [205], via an acoustic analysis module [309], the voice input into a plurality of acoustic features;
mapping [207] the plurality of acoustic features, wherein said mapping [207] comprises identification of a plurality of personality traits based on the acoustic features segmented, followed by determining a corresponding chakra level based on the personality traits mapped, using chakra-based resonance mapping;
aggregating the scores from the mapping and preparing a multidimensional Emotional Quotient (EQ) profile [209] inferred from the said scores; and
generating [211] a structured text-based report [150] summarizing EQ parameters and personality traits associated with the user [110] based on the multidimensional EQ profile.

2. The method [200] as claimed in claim 1, wherein said method [200] further comprises normalizing the voice input received as is, and in view of the contextual metadata.

3. The method [200] as claimed in claim 1, said method [200] further comprising:
capturing at least one biometric parameter of the user from a biometric device to validate energetic states associated with the segmented acoustic features; and
preparing the multidimensional EQ profile further based on the biometric parameters.

4. The method [200] as claimed in claim 1, wherein said segmenting [205] comprises splitting [205a] the voice input into a plurality of discrete acoustic frequency blocks, and extracting [205b] the plurality of acoustic features from said frequency blocks split.

5. The method [200] as claimed in claim 1, wherein the steps of mapping [207], aggregating and preparing [209] the multidimensional EQ profile is performed by an inference and aggregation module [311] that utilizes a Large Language Model (LLM) constrained by a prompt template enforcing interpretation grounded strictly in the acoustic features and the captured contextual metadata.

6. The method [200] as claimed in claim 1, said method [200] further comprising suggesting personalized wellness recommendations including chants, mantras, and wellness practices based on detection of at least a dormant or an active energy state(s) within the EQ profile.

7. A voice-based assessment system [300] for inferring personality, well-being and emotional intelligence (EQ) parameters of a user [110], the system [300] comprising:
an audio input module [305] configured to receive a voice input from the user [110] and normalize the voice input;
a context acquisition module [307] configured to capture a plurality of contextual metadata associated with the voice input and the user [110], said contextual metadata comprising at least one of a time of day, a gender, an age, and a regional location;
an acoustic analysis module [309] configured to segment the voice input into discrete acoustic features;
an inference and aggregation module [311] configured for mapping [207] the plurality of acoustic features, such that said module [311] first identifies a plurality of personality traits based on the acoustic features segmented, and then, determines a corresponding chakra level based on the personality traits mapped, using chakra-based resonance mapping,
wherein further, said module [311] is configured to aggregate the scores from the mapping and prepare [209] a multidimensional Emotional Quotient (EQ) profile; and
an output module [313] configured to generate [211] a structured text-based report [150] summarizing EQ parameters and personality traits associated with the user [110], based on the multidimensional EQ profile.

8. The system [300] of as claimed in claim 7, wherein the output module [313] is configured to convert the determined multidimensional EQ profile into a human-readable artifact in a format selected from Markdown, HTML, or PDF.

9. The system [300] of as claimed in claim 7, further comprising an interactive follow-up module utilizing an agentic workflow to maintain chat history and answer user queries grounded specifically in the generated report [150].

10. The system [300] as claimed in claim 7, wherein the acoustic analysis module [309] is further configured to compute a breath/silence ratio and a fundamental frequency (F0) range to distinguish between different emotional intensities within the voice input.
